(19) United States
(12) Patent Application Publication
(10) Pub. No.: US 2008/0215006 A1
(43) Pub. Date: Sep. 4, 2008

(54) Medical Device with Transcutaneous Cannula Device

(75) Inventor: Ahm Thorkild, Allerod (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvared (DK)

(21) Appl. No.: 11/663,048

(22) PCT Filed: Sep. 22, 2005
(86) PCT No.: PCT/EP2005/054752

§ 371 Date: Nov. 15, 2007

(30) Foreign Application Priority Data
Sep. 22, 2004 (DK) PA 2004 01434

(51) Int. Cl. A61M 5/142; A61M 5/14

(52) U.S. Cl. 604/151; 604/131

(57) ABSTRACT

The present invention generally relates to the insertion of a transcutaneous device of the type comprising a cannula (651) and a therein moveably arranged insertion needle (661), as well as the connecting of such a transcutaneous device with a fluid supply. Thus, a device is provided comprising an insertion needle and a cannula disposed on and being axially moveable relative to the insertion needle, the insertion needle comprising a proximal fluid inlet, a seal (655) being provided between the cannula and the insertion needle allowing fluid to be transported from the fluid inlet to the distal fluid outlet, wherein the insertion needle after having been used to insert the cannula is arranged at a retracted position proximally of the initial position, thereby allowing the fluid inlet to be connected to a fluid supply when it is moved from its initial to its retracted position.

The present invention generally relates to the insertion of a transcutaneous device, especially of the type comprising a cannula and a therein moveably arranged insertion needle, as well as the connecting of such a transcutaneous device with a fluid supply.

### BACKGROUND OF THE INVENTION

In the disclosure of the present invention reference is mostly made to the treatment of diabetes by injection or infusion of insulin, however, this is only an exemplary use of the present invention.

Portable drug delivery devices for delivering a drug to a patient are well known and generally comprise a reservoir adapted to contain a liquid drug and having an outlet in fluid communication with a hollow infusion needle, as well as expelling means for expelling a drug out of the reservoir and through the skin of the subject via the hollow needle. Such devices are often termed infusion pumps.

Basically, infusion pumps can be divided into two classes. The first class comprises durable infusion pumps which are relatively expensive pumps intended for 3-4 years use, for which reason the initial cost for such a pump often is a barrier to this type of therapy. Although more complex than traditional syringes and pens, the pump offer the advantages of continuous infusion of insulin, precision in dosing and optionally programmable delivery profiles and user actuated bolus infusions in connections with meals.

Addressing the above problem, several attempts have been made to provide a second class of drug infusion devices that are low in cost and convenient to use. Some of these devices are intended to be partially or entirely disposable and may provide many of the advantages associated with an infusion pump without the attendant cost and inconveniencies, e.g. the pump may be prefilled thus avoiding the need for filling or refilling a drug reservoir. Examples of this type of infusion devices are known from U.S. Pat. Nos. 4,340,048 and 4,552,561 (based on osmotic pumps), U.S. Pat. No. 5,858,001 (based on a piston pump), U.S. Pat. No. 6,280,148 (based on a membrane pump), U.S. Pat. No. 5,957,895 (based on a flow restrictor pump (also know as a bleeding hole pump)), U.S. Pat. No. 5,527,288 (based on a gas generating pump), or U.S. Pat. No. 5,814,020 (based on a swellable gel) which all in the last decades have been proposed for use in inexpensive, primarily disposable drug infusion devices, the cited documents being incorporated by reference.

The disposable pumps generally comprises a skin-contacting mounting surface adapted for application to the skin of a subject by adhesive means, and with the infusion needle arranged such that in a situation of use it projects from the mounting surface to thereby penetrate the skin of the subject, whereby the place where the needle penetrates the skin is covered while the appliance is in use.

The infusion needle may be arranged to permanently project from the mounting surface such that the needle is inserted simultaneously with the application of the infusion pump. Examples of this configuration can be found in U.S. Pat. Nos. 2,605,765, 4,340,048 and in EP 1 177 802. Although this configuration provides a simple and cost-effective solution, the actual user-performed piercing of the tissue with the needle is often problematic as people who are not experts in medicine are usually insufficiently practised to place such a needle correctly and they often suffer from a fear of the likely pain. Although not relating specifically to infusion pumps, U.S. Pat. No. 5,851,197 discloses an injector in which an infusion set comprising a skin-mountable surface with a protruding needle can be mounted, the injector upon actuation driving the entire infusion set into contact with a skin portion whereby the needle is inserted through the skin.

Addressing the above problem, infusion pump devices have been proposed in which the pump device is supplied to the user with the needle in a retracted state, i.e. with the distal pointed end of the needle “hidden” inside the pump device, this allowing the user to place the pump device on the skin without the possibility of observing the needle. When first the needle is hidden, at least some of the fear is overcome making the introduction of the needle in a second step less problematic. U.S. Pat. Nos. 5,858,001 and 5,814,020 disclose infusion devices of this type in which an infusion needle is arranged in an upper housing portion pivotably arranged relative to a base plate portion. In this way the user can introduce the needle by pressing the upper portion into engagement with the base plate portion.

To further reduce the fear and pain associated with the introduction of the needle, many recent pump devices have been provided with actuatable needle insertion means, which just has to be released by the user after which e.g. spring means quickly will advance the needle through the skin.

For example, U.S. Pat. No. 5,957,895 discloses a liquid drug delivery device comprising a bent injection needle which is adapted to project through a needle aperture in the bottom surface of the housing in a situation of use. A movable needle carrier is disposed in the housing for carrying the injection needle and for causing the injection end of the needle to project through the needle aperture upon movement of the needle carrier.

U.S. Pat. No. 5,931,814 discloses an infusion device having a housing with a drug reservoir, an infusion needle (or cannula) communicating with the reservoir, means for inserting the needle, and pump means for discharging the reservoir contents through the needle. The needle is fixed relative to the housing and projects beyond the lower skin-contacting surface of the housing to the depth required for injection. The needle is surrounded by a protective element which is moved by spring means from a first end position in which the protective device projects beyond the lower surface of the housing and beyond the needle to a second end position in which the protective device does not project beyond the underside of the casing. WO 02/15965 discloses a similar infusion device in which a base plate member acts as a protecting element until an upper part of the device, to which the needle is fixed, is moved down into engagement with the base plate member.

In the devices disclosed in U.S. Pat. Nos. 5,957,895 and 5,931,814 the needle is automatically inserted by the release of pre-tensioned spring means arranged within the devices, whereas in the device known from WO 02/15965 the needle is inserted by the user actively moving the hidden needle. Although the automatic needle insertion means adds convenience for the user and may serve to overcome needle fear, such means also adds to the complexity and thus to the cost of the device, they may reduce the reliability, just as they may add to the bulkiness of the device.

Whereas the above-discussed skin-mountable infusion devices comprise an insertable needle, WO 03/090509 discloses a skin-mountable fluid delivery device comprising an insertable flexible cannula in combination with a therein slidably arranged insertion needle, wherein a seal is provided between the needle and the cannula allowing a fluid to be supplied to the cannula through the needle.

Having regard to the above-identified prior art devices, it is an object of the present invention to provide a medical device comprising a transcutaneous fluid transport device of the type including a cannula and a therein moveably arranged insertion needle, wherein the transcutaneous device is adapted to for easy connection to a fluid supply. The device should be compact in size and be designed for cost effective manufacturing. It is a further object of the invention to provide a fluid transport device which is safe in use. Further objects and advantages of the present invention will be apparent from the below disclosure as well as from the description of exemplary embodiments.

### DISCLOSURE OF ASPECTS OF THE INVENTION

In the disclosure of the present invention, embodiments will be described which will address one or more of the above objects or which will address objects apparent from the below disclosure as well as from the description of exemplary embodiments.

Correspondingly, a medical device is provided, comprising a lower surface adapted for application towards the skin of a subject, a transcutaneous fluid transport device having a distal end portion adapted to be arranged through the skin of the subject and having a distal fluid outlet, and a fluid inlet portion in fluid communication with the distal end portion. The lower surface may be generally planar or it may have another suitable configuration. The fluid transport device comprises an insertion needle and a cannula disposed on (or in) and being axially moveable relative to the insertion needle, the cannula and insertion needle further being moveable relative to the lower surface. The insertion needle comprises a distal end adapted to penetrate the skin of the subject, and a fluid inlet arranged proximally from the cannula, the cannula forming the distal end portion. A seal is provided between the cannula and the insertion needle (i.e. on the inner or outer side of the cannula) allowing fluid to be transported from the fluid inlet to the distal fluid outlet preventing an escape of fluid between the needle and the cannula. In accordance with the invention the fluid transport device has a number of states. More specifically, an initial state in which the cannula and the insertion needle are retracted relative to the lower surface, the fluid inlet being arranged at an initial position, an intermediate state in which the cannula and the insertion needle are extended relative to the lower surface with the distal end of the insertion needle projecting relative to the distal end of the cannula thereby allowing the fluid transport device to be introduced through the skin of the subject, and an extended state in which the cannula extends relative to the lower surface with the distal end of the insertion needle being retracted relative to the distal end of the cannula, the fluid inlet being arranged at a retracted position proximally of the initial position. The insertion needle may be retracted relative to the distal end of the cannula (e.g. the insertion needle may be retracted relative to the lower surface, or the cannula may be extended without the insertion needle being extended at the same speed) before the cannula has been fully extended relative to the housing, this allowing a blunt cannula to serve as the leading element during insertion through a portion of the sub-cutis, this potentially causing less damage.

As appears, this arrangement allows the fluid inlet to be connected to a fluid supply when it is moved from its initial to its retracted position, thereby allowing the insertion needle in a simple way to serve also as a fluid communication between the moveable cannula and the fluid supply. By connecting the fluid inlet of the insertion needle to the fluid supply after the insertion needle has been utilized to insert the cannula, it is provided that the insertion needle can move freely during insertion without being connected to an additional structure.

By the term “towards” is defined that the device may be applied to a skin surface of a subject either directly or indirectly, the latter being the case if the device is mounted on a structure which is adapted for application directly to a skin surface. When the medical device is intended for application directly to a skin surface, the lower surface would be a mounting surface adapted for application on the skin of the subject, the surface being provided with adhesive means (e.g. a medical grade adhesive) for securing the mounting surface to the skin.

In an exemplary embodiment the insertion needle has a pointed or cutting proximal end with the fluid inlet being arranged in the vicinity thereof, and a distal fluid outlet arranged distally of the seal, a fluid conduit being provided therebetween, i.e. the needle being hollow at least between these two openings. By providing a pointed or cutting distal end, the insertion is adapted for connection to a needle-penetratable self-sealing septum, e.g. of the type traditionally used on drug cartridges adapted to be accessed by a traditional hypodermic needle, however, the proximal end may be provided with any desirable configuration allowing it to be connected to a fluid supply. The insertion needle may be in the form of hollow needle comprising a bore therethrough, the fluid inlet and outlet thereby being formed corresponding to the pointed ends.

One of the objects when using a cannula and insertion needle instead of a traditional metallic needle is to provide a transcutaneous device with improved wearing comfort after it has been introduced through the skin. Correspondingly, as the cannula is supported by a relatively stiff insertion needle during insertion through the skin, the cannula can be relatively thin-walled and thus flexible and “soft” in order to accommodate movements between the skin and the skin-mounted device. Although the terms “flexible” and “soft” are relative terms, these are the terms normally used to describe cannulas in the technical field of the present invention. For a given combination of a cannula and an insertion needle the cannula may be described as more flexible than the insertion needle per se (i.e. taking into consideration the materials and the configurations). Indeed, the actual properties of a given cannula should prevent kinking and collapse during the intended use of the cannula. Typically, cannulas for medical use are made from a suitable polymeric material, most of which are flexible thermoplastics e.g. made from or comprising Teflon® or similar, and insertion needles are made from a medical grade stainless steel alloy. A cannula may also be referred to as a catheter.

The length of the transcutaneous device may be chosen in accordance with the actual application, e.g. for insertion at a substantially right angle relative to the skin surface an inserted length of 4-8 mm may be used. However, the cannula may also be inserted at an oblique angle relative to the skin surface for which reason it may be somewhat longer, e.g. 4-20 mm. In order to provide a compact device, exemplary embodiments comprise a deflecting structure, whereby the distal portions of the cannula and the insertion needle are deflected relative to the proximal portions thereof as the cannula and the insertion needle are moved from the retracted to the projecting position. In this way at least a portion of the fluid transport device can be arranged substantially in parallel with the lower surface. Indeed, when a deflecting structure is incorporated, the flexibility of the cannula and, especially, of the insertion needle should be selected in accordance herewith. The needle may e.g. be manufactured from a medical grade polymer or a metal alloy, e.g. stainless steal.

Advantageously the fluid transport device has a further, retracted state in which the cannula and the insertion needle are retracted relative to the lower surface, this allowing a user to retract the cannula before the device is removed from the skin surface and thereby to avoid potential contamination from the exposed, used cannula.

When the insertion needle is arranged outside the soft cannula it may be possible to use a cannula having a smaller outer diameter as it no longer have to accommodate an insertion needle. Thus, in an aspect of the invention, a medical device is provided comprising a housing adapted for application towards the skin of a subject, a cannula having a distal end portion adapted to be arranged through the skin of the subject and having a distal opening, and a needle arranged coaxially with and being axially moveable relative to the cannula, the needle comprising a distal end adapted to penetrate the skin of the subject, wherein the medical device is transformable between a first state in which the cannula and the needle are retracted within the housing, and a second state in which the cannula and the needle are extended relative to the lower surface with the distal end of the needle projecting relative to the distal opening of the cannula thereby allowing the cannula to be introduced through the skin of the subject, wherein the needle is hollow and arranged outside the cannula. Advantageously, the needle may be fully retractable with the cannula in a partly or fully extended position.

The medical device may be in the form of a platform further comprising a coupling for releasably securing the medical device to a mating structure comprising a fluid supply. This arrangement would allow the device to be used as e.g. as an insertion set in combination with an infusion pump, or as a cannula unit in combination with a pump unit.

The medical device may also be in the form of a drug delivery device further comprising drug delivery means including a reservoir adapted to contain a liquid drug, a fluid outlet adapted to be arranged in fluid communication with the fluid inlet of the insertion needle, and expelling means for, in a situation of use, expelling a drug out of the reservoir and through the skin of the subject via the fluid transport device.

The medical device may also form a cannula unit to be used in combination with a pump unit to thereby form a modular drug delivery device or system. A pump unit for such an application would comprise a reservoir adapted to contain a liquid drug, a fluid outlet adapted to be arranged in fluid communication with the fluid inlet of the insertion needle, and expelling means for, in a situation of use, expelling a drug out of the reservoir and through the skin of the subject via the fluid transport device, wherein the medical device and the pump unit comprise mating coupling means allowing the pump unit to be releasable attached to the medical device.

The fluid transport device may be inserted manually, or the medical device may comprise actuatable driving means disposed within the housing and adapted to provide the necessary translation of the cannula and the insertion needle. The reservoir may be pre-filled or it may be adapted to be filled by the user prior to use.

For the above embodiments it has been described that the cannula may be retracted relative to the lower surface after use, however, this feature may find broad application for a medical device comprising a transcutaneous fluid transport device of the type including a cannula and a therein moveably arranged insertion needle. Correspondingly, in a further aspect a medical device is provided comprising a lower surface adapted for application towards the skin of a subject, and a transcutaneous fluid transport device having a distal end portion adapted to be arranged through the skin of the subject and having a distal fluid outlet, and a fluid inlet portion in fluid communication with the distal end portion. The fluid transport device comprises an insertion needle and a cannula disposed on and being axially moveable relative to the insertion needle, the insertion needle comprising a pointed distal end, the cannula forming the distal end portion, wherein the fluid transport device has an initial state in which the cannula and the insertion needle are retracted relative to the lower surface, an actuated state in which the cannula and the insertion needle are extended relative to the lower surface with the distal end of the insertion needle projecting from a distal opening in the cannula thereby allowing the fluid transport device to be introduced through the skin of the subject, an extended state in which the cannula extends relative to the lower surface with the distal end of the insertion needle being retracted from the distal opening in the cannula, and a retracted state in which the cannula and the insertion needle are retracted relative to the lower surface.

For such a device the fluid inlet may be provided in either the cannula or the needle (using the above-described seal), just as the fluid connection with the fluid supplying structure may be provided by any suitable structure, for example as described in WO 03/090509.

The present invention also provides a method comprising the steps of providing a medical device having a housing with a lower surface, a reservoir, and a transcutaneous fluid transport device comprising a hollow insertion needle and a cannula disposed on and being axially moveable relative to the insertion needle, the insertion needle comprising a proximal end and a pointed distal end, actuating the transcutaneous fluid transport device from an initial state in which the cannula and the insertion needle are retracted relative to the lower surface, to an extended state in which the cannula and the insertion needle are extended relative to the lower surface with the distal end of the insertion needle projecting from the cannula, and retracting the insertion needle to a position proximal of the initial position to thereby connect the insertion needle with the reservoir.

The devices described above in accordance with individual aspects of the invention can be used both independently of each other and in combination with elements in accordance with other aspects and features of the invention.

As used herein, the term “drug” is meant to encompass any drug-containing flowable medicine capable of being passed through a delivery means such as a hollow needle in a controlled manner, such as a liquid, solution, gel or fine suspension. Representative drugs include pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form. In the description of the exemplary embodiments reference will be made to the use of insulin. Correspondingly, the term “subcutaneous” infusion is meant to encompass any method of transcutaneous delivery to a subject. Further, the term needle (when not otherwise specified) defines a piercing member adapted to penetrate the skin of a subject.

### BRIEF DESCRIPTION OF THE DRAWINGS

In the following the invention will be further described with references to the drawings, wherein

In the following the invention will be further described with references to the drawings, wherein

FIGS. 1-11 shows in perspective views the sequences of use of the present invention embodied in a drug delivery device,

FIG. 12 shows a further embodiment of a reservoir unit,

FIGS. 13A-13C show in a schematic representation an embodiment of a transcutaneous device in the form of a cannula and insertion needle combination,

FIGS. 14A and 14B show in a schematic representation a transcutaneous device in the form of a cannula and insertion needle combination,

FIG. 15 shows in an exploded perspective view a reservoir unit,

FIGS. 16A-16E show different expelling means suitable for use with drug delivery devices incorporating the present invention,

FIGS. 17A-17C show in a schematic representation an embodiment of a transcutaneous device in the form of a cannula and insertion needle combination,

FIGS. 18A and 18B show embodiments of a transcutaneous device, and

FIGS. 19A and 19B show cross-sectional views of embodiments of a soft cannula.

In most of the figures like structures are identified by like reference numerals.

### DESCRIPTION OF EXEMPLARY EMBODIMENTS

When in the following terms as “upper” and “lower”, “right” and “left”, “horizontal” and “vertical” or similar relative expressions are used, these only refer to the appended figures and not to an actual situation of use. The shown figures are schematic representations for which reason the configuration of the different structures as well as there relative dimensions are intended to serve illustrative purposes only.

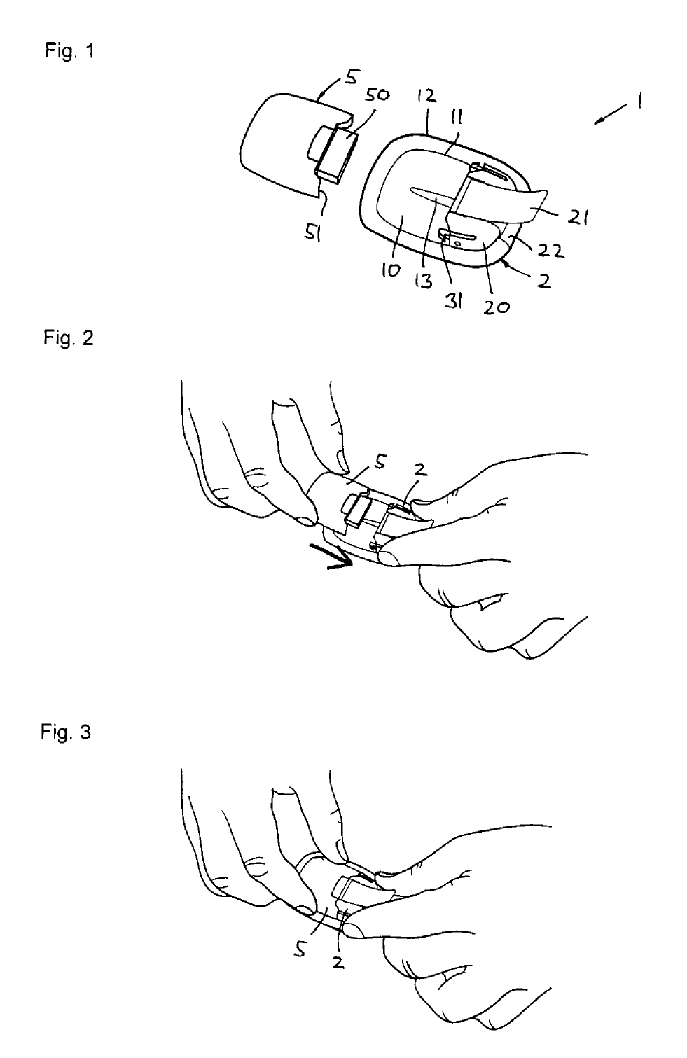

Firstly, with reference to FIGS. 1-12 an embodiment of a drug delivery device will be described focusing primarily on the directly user-oriented features. The delivery device is shown as an example of a type of device in which the present invention advantageously may be implemented, however, the described modular delivery can be considered to be “generic” in respect of the transcutaneous device actually used, e.g. a needle or a cannula.

The transcutaneous device unit 2 comprises a transcutaneous device in the form of a cannula and an associated insertion needle and will thus in the following be termed a cannula unit.

More specifically, FIG. 1 shows a perspective view of medical device in the form of a modular skin-mountable drug delivery device 1 comprising a patch-like cannula unit 2 and a reservoir unit 5. When supplied to the user each of the units are preferably enclosed in its own sealed package (not shown).

The cannula unit comprises a base portion 10 with a lower mounting surface adapted for application to the skin of a user, and a housing portion 20 in which a hollow infusion cannula (not shown) is arranged. The cannula comprises a distal portion adapted to penetrate the skin of a user, and a proximal portion adapted to be arranged in fluid communication with the reservoir unit. The distal portion of the cannula is moveable between an initial position in which the distal end is retracted relative to the mounting surface, and an extended position in which it projects relative to the mounting surface. Further, the cannula is moveable between the extended position in which the distal end projects relative to the mounting surface, and a retracted position in which the distal end is retracted relative to the mounting surface. The cannula unit further comprises user-gripable actuation means in the form of a first strip-member 21 for moving the distal end of the cannula between the initial and the second position when the actuation means is actuated, and user-gripable retraction means in the form of a second strip-member 22 means for moving the distal end of the cannula between the extended and the retracted position when the retraction means is actuated. As can be seen, the second strip is initially covered by the first strip. The housing further comprises user-actuatable male coupling means 40 in the form of a pair of resiliently arranged hook members adapted to cooperate with corresponding female coupling means on the reservoir unit, this allowing the reservoir unit to be releasable secured to the cannula unit in the situation of use. In the shown embodiment the base portion comprises a relatively rigid upper portion 11 attached to a more flexible adhesive sheet member 12 having a lower adhesive surface providing the mounting surface per se, the adhesive surface being supplied with a peelable protective sheet. The base portion also comprises a ridge member 13 adapted to engage a corresponding groove on the reservoir unit.

The reservoir unit 5 comprises a pre-filled reservoir containing a liquid drug formulation (e.g. insulin) and expelling means in the form of an electronically controlled pump for expelling the drug from the reservoir through the cannula in a situation of use. The reservoir unit has a generally flat lower surface adapted to be mounted onto the upper surface of the base portion, and comprises a protruding portion 50 adapted to be received in a corresponding cavity of the housing portion 20 as well as female coupling means 51 adapted to engage the corresponding hook members 31 on the cannula unit. The protruding portion provides the interface between the two units and comprises a pump outlet and contact means (not shown) allowing the pump to be started as the two units are assembled. The lower surface also comprises a window (not to be seen) allowing the user to visually control the contents of the reservoir.

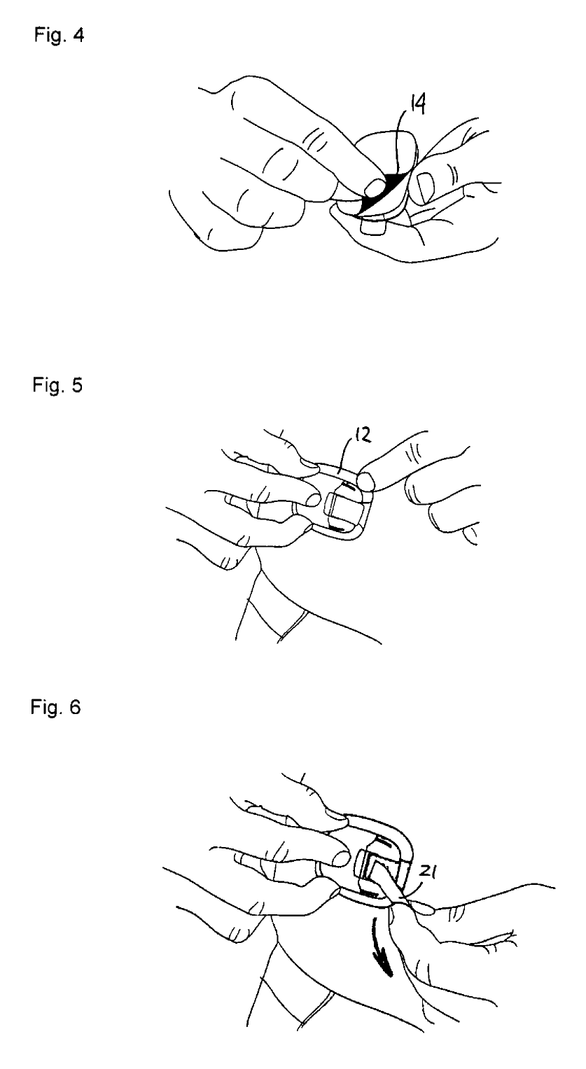

First step in the mounting procedure is to assemble the two units by simply sliding the reservoir unit into engagement with the cannula unit (FIG. 2). When the hook members properly engage the reservoir unit a “click” sound is heard (FIG. 3) signalling to the user that the two units have been properly assembled. If desired, a visual or audible signal may also be generated. Thereafter the user removes the peelable sheet 14 to uncover the adhesive surface (FIG. 4) where after the device can be attached to a skin surface of the user, typically the abdomen (FIG. 5). Infusion of drug is started by gripping and pulling away the actuation strip 21 as indicated by the arrow whereby the cannula is inserted followed by automatic start of the infusion (FIG. 6). The cannula insertion mechanism may be supplied in a pre-stressed state and subsequently released by the actuation means or the cannula insertion may be “energized” by the user. A “beep” signal confirms that the device is operating and drug is infused. The reservoir unit is preferably provided with signal means and detection means providing the user with an audible alarm signal in case of e.g. occlusion, pump failure or end of content.

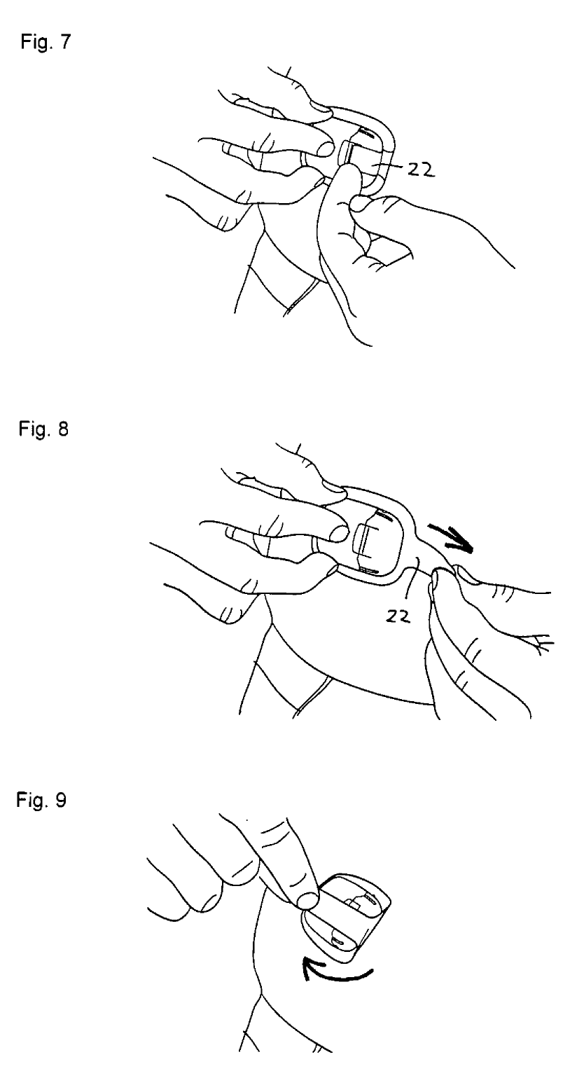

After the device has been left in place for the recommended period of time for use of the cannula unit (e.g. 48 hours)—or in case the reservoir runs empty or for other reasons—it is removed from the skin by gripping (FIG. 7) and pulling (FIG. 8) the retraction strip 22 as indicated by the arrows which leads to retraction of the cannula followed by automatic stop of drug infusion where after the strip which is attached to the adhesive patch is used to remove the device from the skin surface (FIG. 9).

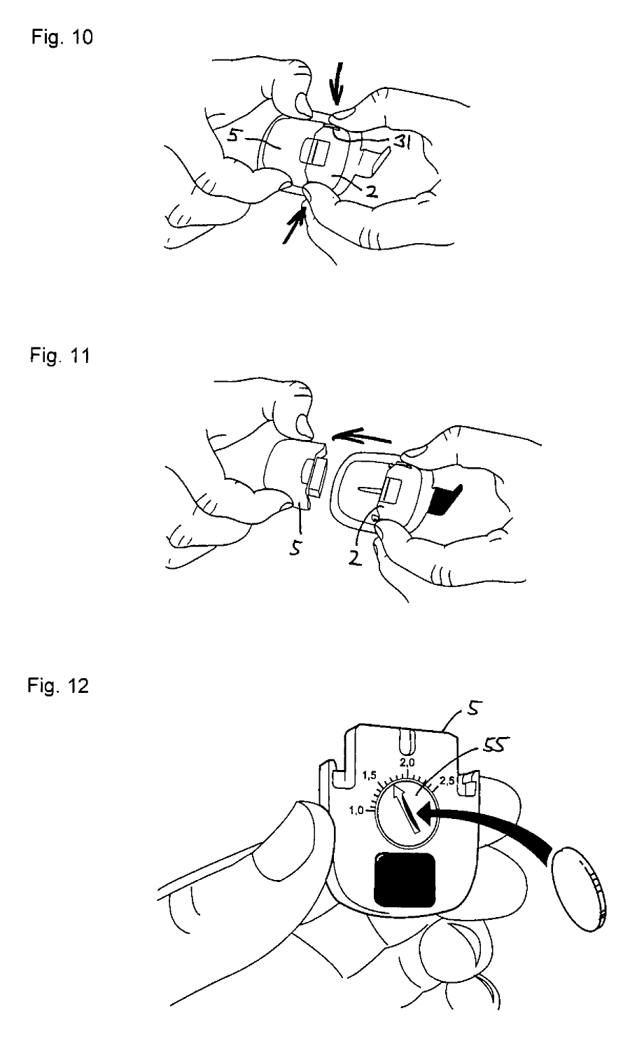

When the device has been removed the two units are disengaged by simultaneously depressing the two hook members 31 as indicated by the arrows (FIG. 10) allowing the reservoir unit 5 to be pulled out of engagement with the cannula unit 2 as indicated by the arrow (FIG. 11) which can then be discarded. Thereafter the reservoir unit can be used again with fresh cannula units until it has been emptied.

The reservoir unit may be supplied with a fixed basal infusion rate or it may be supplied as an adjustable unit (FIG. 12) with adjustment means 55 allowing the infusion rate to be set by a physician and/or the user/patient. The reservoir unit may also be provided with means allowing the control means to be programmed or set electronically (not shown).

The device described with reference to FIGS. 1-11 may also be used in alternative ways. For example, the cannula unit may be mounted to the skin after which the reservoir is attached. Depending on the configuration of the cannula unit, it may be possible or prevented that the cannula is introduced before the reservoir unit is attached.

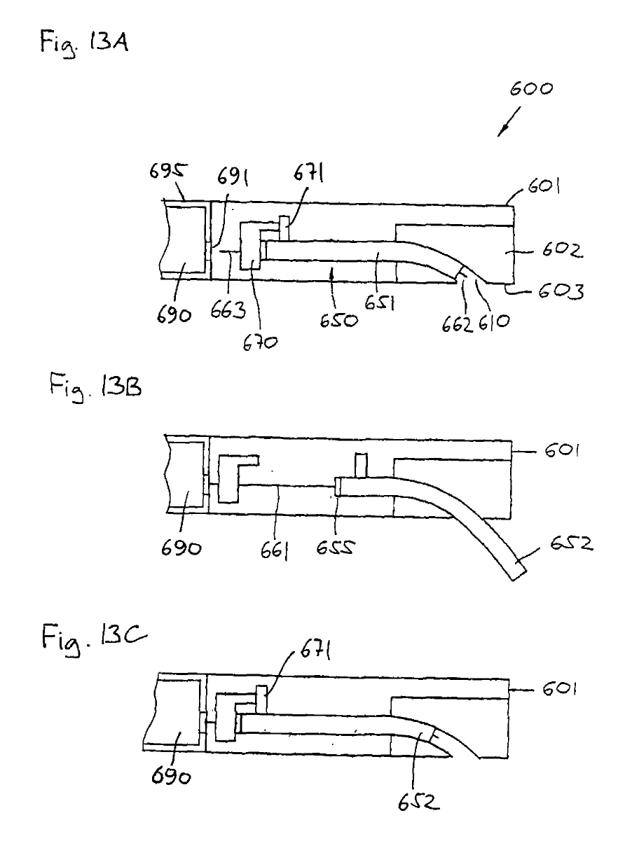

FIGS. 13A-13C show in a schematic representation an embodiment of a cannula and insertion needle combination arranged within a housing 601 of a transcutaneous device unit 600, e.g. corresponding to the cannula unit 2, attached to a reservoir/pump unit 695 (shown partially), e.g. corresponding to the reservoir unit 5, however, the device may also be supplied as a unitary structure. It should be noted that the relative dimensions of the FIG. 13 embodiment are different from those of the cannula unit 2. More specifically, FIG. 13A shows a medical device unit in an initial state comprising a transcutaneous assembly 650 comprising a combination of a relatively rigid, hollow insertion needle 661 (e.g. made from medical grade stainless steel) and a relatively soft cannula 651 (which e.g. may be of the soft “Teflon®” type) disposed on the insertion cannula. The cannula includes a seal portion 655 which enables the cannula and the needle to move relative to each other while maintaining a seal therebetween. The seal portion may be arranged at the end of the cannula or inside the cannula, and may be provided by structures on either the cannula, e.g. a constriction, or the needle, e.g. a larger-diameter portion, or on both. The insertion needle is coupled to an actuation member 670 and has a pointed distal end 662 which in the initial state protrudes from a distal opening in a distal portion 652 of the cannula, and a proximal end 663 adapted to be arranged in fluid communication with a fluid providing structure 690, e.g. a conduit or directly with a pump or a fluid reservoir. In the shown embodiment the needle comprises a pointed proximal end which connects to the fluid supply via a needle-penetratable elastomeric septum 691. The housing 601 further comprises a lower surface 603 with an exit opening 610 for the cannula.

In a situation of use the transcutaneous device unit (or the device in which it is incorporated) is arranged on a skin surface of a subject and the actuation member 670 which can then be moved from an initial (or first) state to an actuated state by which both the cannula and the insertion needle is moved to an extended position (the latter being pushed by the actuation member) by which action the combined distal ends of the cannula and the insertion needle is advanced through the exit opening and subsequently through the skin of the subject. In this position the cannula is locked in place (either reversibly or irreversibly), e.g. by friction or by additional locking means (not shown), where after the insertion needle is withdrawn proximally as shown in FIG. 13B by moving the actuation member backwardly, however, the needle is still in sealed fluid communication with the cannula. As seen, the insertion needle is withdrawn further proximally compared to its initial position whereby the proximal end of the insertion needle penetrates the septum member 691, this allowing connection to the fluid source housed in the reservoir/pump unit. The transcutaneous assembly may be moved by actuation means from the outside, e.g. manually, however, energized actuation means (e.g. one or more spring members) may be included within the housing and adapted to be released by external means such as the strip member 21, see FIG. 6. Examples of actuation means suitable for the actuation of a cannula needle assembly as described above is described in WO 03/090509 and WO 02/40083 which are hereby incorporated by reference. After use, the cannula may be retracted into the device (see FIG. 13C) before the device is removed from the skin of the subject. To this order the cannula is provided with a retraction member 671 which may be engaged either directly by the user or via a release mechanism as shown in FIG. 8.

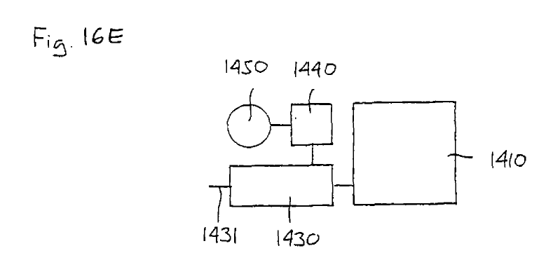

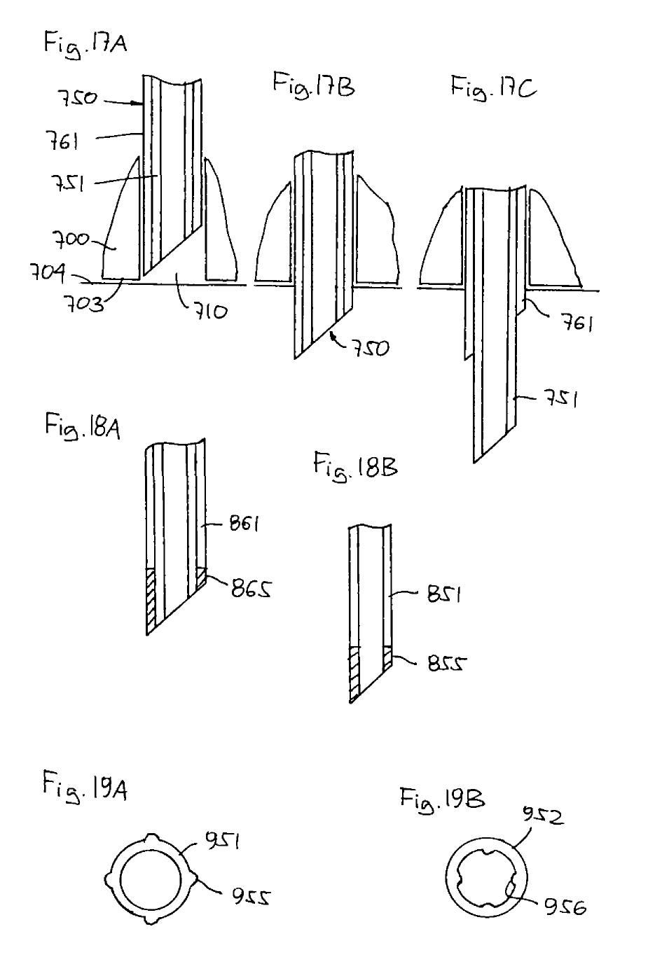

In FIGS. 13A-13C an embodiment with an insertion needle arranged within a soft cannula is shown, however, the insertion needle may also be arranged outside the soft cannula. More specifically, FIGS. 17A shows in a schematic representation a distal portion of a transcutaneous assembly 750 comprising a combination of an outer hollow insertion needle 761 with a cutting distal end and a co-axially arranged inner relatively soft cannula 751 (which e.g. may be of the soft “Teflon®” type) disposed within the insertion cannula, the transcutaneous assembly being arranged within a skin-mountable device 700 having a lower surface 703 adapted for application towards the skin of a subject and comprising an opening 710 for the transcutaneous assembly. In the shown embodiment the distal ends of both the insertion needle and the cannula are obliquely cut and aligned with each other. When the transcutaneous assembly is moved from its initial position as shown in FIG. 17B the distal cutting edge of the insertion needle with penetrate the skin of the subject. From this point the soft cannula may be advanced further to its fully extended position “on its own” through the subcutaneous tissue of the subject as shown in FIG. 17C, or the assembly may be fully introduced as a unit after which the insertion needle is withdrawn. If the insertion needle is used only to penetrate the outer denser layer of the skin, the soft cannula will have to be provided with a columnar strength and a distal end portion allowing it to be introduced through the softer subcutaneous tissue. In contrast, if the soft cannula is arranged within the outer insertion needle during the entire insertion procedure, a high degree of columnar strength is less important. In the shown embodiment the transcutaneous assembly is straight allowing a rigid outer insertion needle to be used, however, if the transcutaneous assembly is curved as shown in FIGS. 13A the outer insertion needle will either have to be flexible or it will have to be short and arranged only along a portion of the soft cannula, e.g. as a ring or short tube, the latter arrangement allowing the insertion needle to penetrate only the superficial layers of the skin of the subject as shown in FIG. 17B. A straight transcutaneous assembly may be inserted perpendicularly relative to a skin surface as shown in FIGS. 17A-17C or it may be inserted at an angle, e.g. corresponding to an angled insertion as shown in FIG. 13B. In order to fixate the soft cannula relative to the device in the extended position, the soft cannula will e.g. have to extend proximally relative to the insertion needle or a longitudinal opening has to be provided in the outer insertion needle, this allowing the soft cannula to be engaged by a gripping structure. If it is desirable to use a flexible outer insertion needle, such a needle may be provided with a distal end portion formed from a material adapted for providing a sharp cutting edge, e.g. an insertion needle 861 may be formed from a relatively soft polymer and the distal portion 865 comprising the cutting edge may be formed from a harder polymer or a metal alloy, this as shown in FIG. 18A. Using the same principle, a soft cannula 851 as shown in FIG. 18B may correspondingly comprise a distal tip 855 with a cutting distal edge formed from a different material, this allowing the soft cannula to be introduced without an insertion needle given the necessary columnar strength. A higher columnar strength or a greater resistance to kinking for a soft cannula of a given material may be achieved by a larger outer diameter or a larger wall thickness, however, it is normally desirable to have a small outer diameter and a large bore. To provide a higher columnar strength or a greater resistance to kinking a configuration different from circular may be used, e.g. oval or angular. In addition or alternatively a soft cannula 951, 952 may be provided with ribs 955, 956 on the inner or outer surface, or both, at least along a portion of the length of the cannula, see FIGS. 19A and 19B.

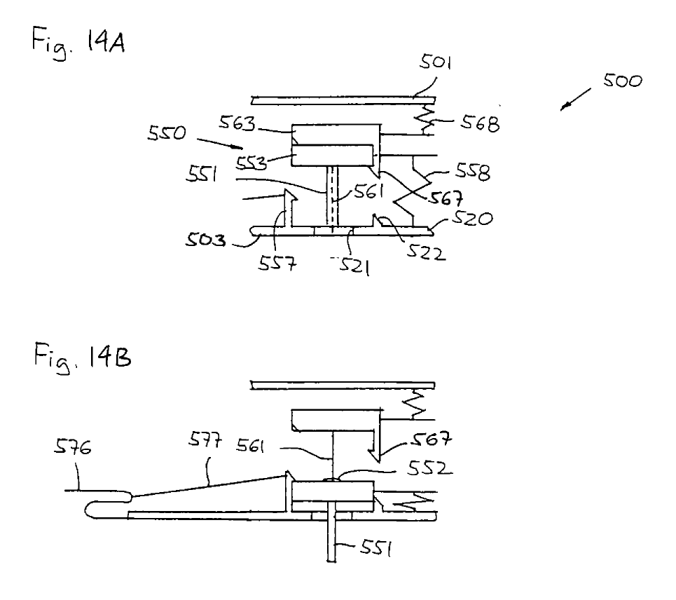

FIGS. 14A and 14B show in a further schematic representation how a retractable cannula and insertion needle combination can be arranged within a housing 501 of a given medical device 500 (partly shown), e.g. a cannula unit as shown in FIG. 1, a unitary drug delivery device or an infusion set, the device comprising a lower surface 503. More specifically, the medical device comprises a transcutaneous assembly 550 comprising a combination of a relatively soft cannula 551 (which e.g. may be of the soft “Teflon®” type) carried by a lower member 553 and a pointed insertion needle 561 (e.g. made from medical grade stainless steel) slidably arranged within the cannula and carried by an upper member 563, both members being mounted to allow axial displacement of the cannula respectively the insertion needle. The cannula is in flow communication with a proximal inlet (not shown) allowing it to be or to be arranged in fluid communication with a fluid source. The medical device further comprises a base plate 520 with an opening 521 for the cannula as well as a release member 522. The lower member comprises an elastomeric seal 552 through which the insertion needle is arranged. The cannula and the insertion needle may be straight or curved dependent upon how the two members are mounted in the device, e.g. arcuate corresponding to a pivoting axis or straight corresponding to linear movement as illustrated. The upper member comprises a coupling member 567 locking the members together in an initial position with the distal end of the insertion needle extending from the distal opening of the cannula as shown in FIG. 14A, and the base plate comprises coupling member 557 for locking the lower member in an extended position with distal end of the cannula extending through the opening in the base plate (see FIG. 14B). Between the housing of the device and the upper member a first spring 568 is arranged biasing the upper member upwards. Correspondingly, the device also comprises a second spring 558 biasing the lower member upwardly. The medical device further comprises a gripping tab 576 and a pulling member 577 corresponding to the embodiment shown in FIG. 1.

In a situation of use the assembly is moved downwardly, either manually, by a pre-stressed assembly (not shown) or by a releasable insertion aid, e.g. a spring loaded member acting through an opening in the housing (not shown) whereby the cannula with the projecting insertion needle is inserted through the skin of a subject. In this position the lower member engages the coupling member 557 to thereby lock the cannula in its extended position, just as the coupling member 567 is released by the release member 522 thereby allowing the upper member to return to its initial position by means of the first spring.

When the user intends to remove the delivery device from the skin surface, the user grips the gripping portion of the tab and pulls it in a first direction substantially in parallel with the skin surface, by which action the flexible strip 577 releases the coupling member 557 from the lower member whereby the lower member and thereby the cannula is retracted by means of the second spring. When the cannula has been withdrawn from the skin, the user uses the now unfolded tab to pull off the entire delivery device from the skin surface, for example by pulling the tab in a direction away from the skin surface.

With reference to FIG. 15 an embodiment of a reservoir unit 405 of a type suitable to be used with a cannula unit is shown, the upper portion of the housing being removed. The reservoir unit comprises a reservoir 460 and an expelling assembly comprising a pump assembly 400 and control and actuation means 480, 481 therefore. The pump assembly comprises an outlet 422 for connection to a transcutaneous access device (e.g. the cannula 650) and an opening 423 allowing a fluid connector housed in the pump assembly to be actuated and connected to the reservoir. The reservoir 460 is in the form of prefilled, flexible and collapsible pouch comprising a needle-penetratable septum adapted to be arranged in fluid communication with the pump assembly, see below. The shown pump assembly is a mechanically actuated membrane pump, however, the reservoir and expelling means may be of any suitable configuration.

The control and actuation means comprises a pump actuating member in the form of a coil actuator 481 arranged to actuate a piston of the membrane pump, a PCB or flex-print to which are connected a microprocessor 483 for controlling, among other, the pump actuation, contacts 488, 489 cooperating with the contact actuators on the needle unit, signal generating means 485 for generating an audible and/or tactile signal, a display (not shown) and an energy source 486. The contacts are preferably protected by membranes which may be formed by flexible portions of the housing.

In the above-described embodiment a reservoir unit or a drug delivery device comprising a reservoir has been described, however, for better illustrating the principles of the present invention, the means for expelling a drug from the reservoir has been omitted in some of the figures. Such expelling means, which as the reservoir does not form part of the present invention in its basic form, may be of any type which would be suitable for arrangement within a skin-mountable drug delivery device or reservoir unit. Further, as the needle of the present invention also may be in the form of a needle sensor, the interior of the corresponding medical device may comprise sensor means adapted to cooperate with the needle sensor.

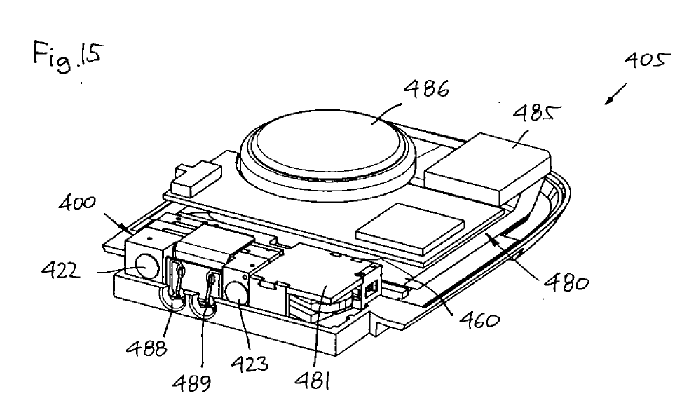

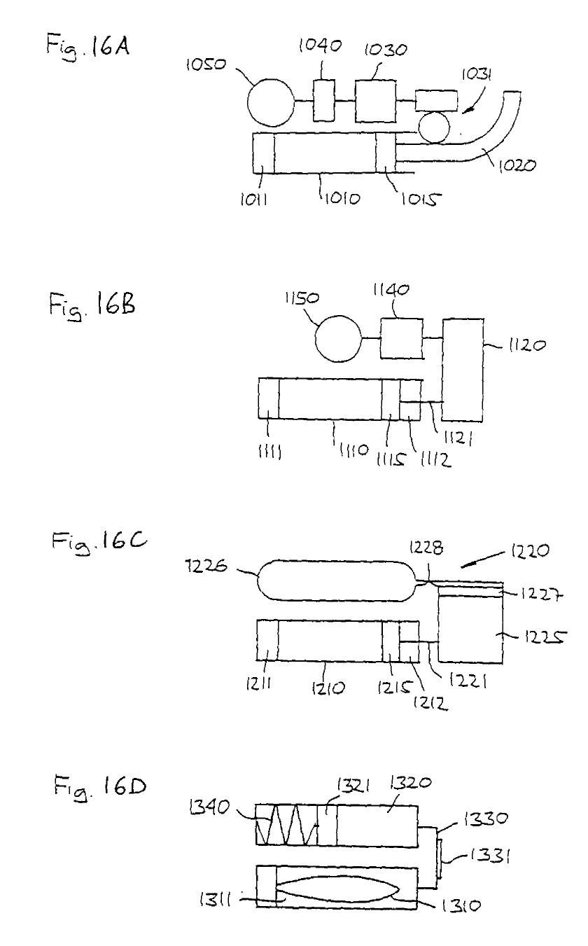

In FIGS. 16A-16E examples of expelling means suitable for use with the present invention are shown schematically, however, these are merely examples, just as the shown arrangement of the individual components not necessarily are suitable for direct application in the above shown delivery devices. More specifically, FIG. 16A shows a pump arrangement comprising a drug-containing cartridge 1010 forming a reservoir and having a distal closure member 1011 allowing a needle to be connected, and a piston 1015 slidingly arranged there within, a flexible toothed piston rod 1020 (for example as disclosed in U.S. Pat. No. 6,302,869), an electric motor 1030 which via a worm-gear arrangement 1031 drives the piston rod to expel drug from the cartridge, the motor being controlled by control means 1040 and the energy for the control means and the motor being provided by a battery 1050. The pump may be activated when the needle is inserted (by means not shown) or by separate user-actuatable means (not shown) after the inserter has been detached form the delivery device.

FIG. 16B shows a pump arrangement comprising a drug-containing cartridge 1110 having distal and proximal closure members 1111, 1112, and a piston 1115 slidingly arranged there within, gas generating means 1120 in fluid communication with the interior of the cartridge via conduit 1121 for driving the piston to expel drug from the cartridge, the gas generating means being controlled by control means 1140 and the energy for the control means and the gas generation being provided by a battery 1150. The pump may be activated as indicated above. A detailed disclosure of such gas generating means for a drug delivery device can be found in e.g. U.S. Pat. No. 5,858,001.

FIG. 16C shows a pump arrangement comprising a drug-containing cartridge 1210 having distal and proximal closure members 1211, 1212, and a piston slidingly 1215 arranged there within, an osmotic engine 1220 in fluid communication with the interior of the cartridge via conduit 1221 for driving the piston to expel drug from the cartridge. The osmotic engine comprises a first rigid reservoir 1225 containing a salt-solution and a second collapsible reservoir 1226 containing water, the two reservoirs being separated by a semi-permeable membrane 1227. When supplied to the user, the fluid connection 1228 between the second reservoir and the membrane is closed by a user-severable membrane (e.g. a weak weld) which, when severed, will allow the osmotic process to start as water is drawn from the second reservoir through the membrane and into the first reservoir. The pump may be activated as indicated above. A detailed disclosure of the osmotic drive principle can be found in e.g. U.S. Pat. No. 5,169,390.

FIG. 16D shows a pump arrangement comprising a drug-containing flexible reservoir 1310 arranged within a rigid fluid-filled secondary reservoir 1311 in fluid communication with a primary reservoir 1320 through a conduit 1330 comprising a flow restrictor 1331. The primary reservoir is in the form of a cartridge with a moveable piston 1321 and contains a viscous drive fluid. A spring 1340 is arranged to act on the piston to drive fluid from the first to the second reservoir thereby expelling drug from the flexible reservoir when the latter is connected to an infusion needle (not shown). The flow rate will be determined by the pressure generated by the spring in the drive fluid, the viscosity of the drive fluid and the flow resistance in the flow restrictor (i.e. bleeding hole principle). The pump may be activated by straining the spring or by releasing a pre-stressed spring, either when the needle is inserted (by means not shown) or by separate user-actuatable means (not shown) after the inserter has been detached form the delivery device. An example of this principle used for drug infusion is known from DE 25 52 446. In an alternative configuration, the drug reservoir may be pressurized directly to expel the drug via a flow restrictor, e.g. as disclosed in U.S. Pat. No. 6,074,369.

FIG. 16E shows a pump arrangement comprising a membrane pump 1430 having an outlet 1431 and control means 1440 for controlling the pump, the energy for the control means and the pump being provided by a battery 1450. The membrane pump is (in a situation of use) connected to a reservoir 1410 from which drug is sucked through the pump and expelled through the outlet. The reservoir may be provided with venting means or it may be in the form of a flexible, collapsible reservoir whereby venting means can be dispensed with. The pump may be activated when the needle is inserted (by means not shown) or by separate user-actuatable means (not shown) after the inserter has been detached form the delivery device.

The above-described transcutaneous assembly is of the same type as described in WO 03/090509 and WO 02/40083 which are hereby incorporated by reference. This document discloses a number of further transcutaneous assemblies which advantageously may be arranged in a sealed structure corresponding to the present invention.

In the above description of the preferred embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A medical device, comprising:
a lower surface (603) adapted for application towards the skin of a subject,
a transcutaneous fluid transport device having a distal end portion (652) adapted to be arranged through the skin of the subject and having a distal fluid outlet, and a fluid inlet portion in fluid communication with the distal end portion, the fluid transport device comprising an insertion needle (661) and a cannula (651) disposed on, or in, and being axially moveable relative to the insertion needle, the insertion needle comprising a distal end (662) adapted to penetrate the skin of the subject, and a fluid inlet (663) arranged proximally from the cannula, the cannula forming the distal end portion, a seal (655) being provided between the cannula and the insertion needle allowing fluid to be transported from the fluid inlet to the distal fluid outlet,
wherein the fluid transport device has:
an initial state in which the cannula and the insertion needle are retracted relative to the lower surface, the fluid inlet being arranged at an initial position,
an intermediate state in which the cannula and the insertion needle are extended relative to the lower surface with the distal end of the insertion needle projecting relative to the distal end of the cannula thereby allowing the fluid transport device to be introduced through the skin of the subject, and
an extended state in which the cannula extends relative to the lower surface with the distal end of the insertion needle being retracted relative to the distal end of the cannula, the fluid inlet being arranged at a retracted position proximally of the initial position,
thereby allowing the fluid inlet to be connected to a fluid supply (690) when it is moved from its initial to its retracted position.

2. A medical device as defined in claim 1, wherein the insertion needle has a pointed or cutting proximal end with the fluid inlet being arranged in the vicinity thereof, a distal fluid outlet arranged distally of the seal and a fluid conduit therebetween.

3. A medical device as defined in claim 2, wherein the insertion needle fluid inlet and outlet are formed corresponding to the ends of the insertion needle, the fluid conduit being formed by a bore along the length of the insertion needle.

4. A medical device as defined in claim 1, wherein the fluid transport device has a further, retracted state in which the cannula and the insertion needle are retracted relative to the lower surface.

5. A medical device as defined in any of claim 1, further comprising a coupling (31) for releasably securing the medical device to a mating structure comprising a fluid supply.

6. A medical device as defined in any of claim 1, further comprising a drug delivery assembly comprising:
a reservoir (460) adapted to contain a liquid drug,
a fluid outlet adapted to be arranged in fluid communication with the fluid inlet of the insertion needle, and
expelling means (470) for, in a situation of use, expelling a drug out of the reservoir and through the skin of the subject via the fluid transport device.

7. A medical device as defined in any of claim 1, in combination with a pump unit (5, 450) comprising:
a reservoir (460) adapted to contain a liquid drug,
a fluid outlet adapted to be arranged in fluid communication with the fluid inlet of the insertion needle, and
expelling means (460) for, in a situation of use, expelling a drug out of the reservoir and through the skin of the subject via the fluid transport device,
wherein the medical device and the pump unit comprise mating coupling means (31, 51) allowing the pump unit to be releasable attached to the medical device.

8. A medical device as defined claim 1, wherein the lower surface is a mounting surface adapted for application on the skin of the subject, the attaching means being adhesive means provided on the mounting surface for securing the mounting surface to the skin.

9. A medical device as defined in claim 1, wherein the cannula is soft and flexible and made from a polymeric material.

10. A medical device, comprising:
a lower surface (503, 603) adapted for application towards the skin of a subject,
a transcutaneous fluid transport device having a distal end portion adapted to be arranged through the skin of the subject and having a distal fluid outlet, and a fluid inlet portion in fluid communication with the distal end portion, the fluid transport device comprising an insertion needle (561, 661) and a cannula (551, 651) disposed on and being axially moveable relative to the insertion needle, the insertion needle comprising a pointed distal end, the cannula forming the distal end portion,
wherein the fluid transport device has:
an initial state in which the cannula and the insertion needle are retracted relative to the lower surface,
an actuated state in which the cannula and the insertion needle are extended relative to the lower surface with the distal end of the insertion needle projecting from a distal opening in the cannula thereby allowing the fluid transport device to be introduced through the skin of the subject,
an extended state in which the cannula extends relative to the lower surface with the distal end of the insertion needle being retracted from the distal opening in the cannula, and
a retracted state in which the cannula and the insertion needle are retracted relative to the lower surface.

11. A method comprising the steps of:
providing a medical device having a housing with a lower surface, a reservoir, and a transcutaneous fluid transport device comprising a hollow insertion needle (561, 661) and a cannula (551, 651) disposed on and being axially moveable relative to the insertion needle, the insertion needle comprising a proximal end and a pointed distal end,
actuating the transcutaneous fluid transport device from an initial state in which the cannula and the insertion needle are retracted relative to the lower surface, to an intermediate state in which the cannula and the insertion needle are extended relative to the lower surface with the distal end of the insertion needle projecting relative to the distal end of the cannula thereby allowing the fluid transport device to be introduced through the skin of the subject, and further to an extended state in which the cannula extends relative to the lower surface with the distal end of the insertion needle being retracted relative to the distal end of the cannula, and
retracting the insertion needle to a position proximal of the initial position to thereby connect the insertion needle with the reservoir.

12. A method as in claim 11, wherein the insertion needle is retracted relative to the distal end of the cannula before the cannula has been fully extended relative to the housing.

13. A method as in claim 11, wherein the needle is arranged either within the cannula or outside the cannula.
